(19) United States
(12) Patent Application Publication
(10) Pub. No.: US 2024/0115203 A1
(43) Pub. Date: Apr. 11, 2024

(54) SWEAT PATCH WITH IMPROVED ADHESION AND ANTI-TAMPERING FEATURE(S)

(71) Applicant: Smart Sales, LLC dba Smartox, Irving, TX (US)

(72) Inventors: Tom A. Anthony, Nichols Hills, OK (US); Christopher D. Nabors, McKinney, TX (US)

(21) Appl. No.: 18/481,789

(22) Filed: Oct. 5, 2023

Related U.S. Application Data
(60) Provisional application No. 63/379,086, filed on Oct. 11, 2022.

(51) Int. Cl. A61B 5/00

(52) U.S. Cl. CPC A61B 5/6833; A61B 5/4266

(57) ABSTRACT

Improved wear time and anti-tampering for a sweat patch is achieved based on the adhesive used to secure the sweat patch to the subject. Two adhesives may be used with one securing regions immediately surrounding the collection pad to the subject's skin, while a second secures an outer ring of the sweat patch to the subject's skin. The second adhesive provides greater adhesion to the skin under common conditions, such as profuse sweating or immersion in water, thereby providing a longer wear time and greater analyte collection integrity as well as one anti-tampering feature. Other anti-tampering features may also be provided.

### PRIORITY CLAIM

This application claims priority to U.S. Provisional Patent Application No. 63/379,086 filed Oct. 11, 2022. The content of the above-identified patent document(s) is incorporated herein by reference.

### TECHNICAL FIELD

This disclosure relates generally to detecting analytes in body fluid. More specifically, this disclosure relates to improving adhesion and tamper resistance of a patch for collecting fluids excreted through the skin of a subject.

### BACKGROUND

Dermal patches for collecting body fluid expressed through the skin by perspiration (“sweat”) are described in U.S. Pat. No. 4,957,108 and its progeny, and U.S. Pat. No. 6,492,180. Such “sweat” patches are commercially available to detect proscribed consumption of alcohol or use of marijuana, cocaine, opiates, amphetamines/methamphetamines, phenylcyclo-hexyl piperidine (a/k/a “PCP”), fentanyl, oxycodone, oxymorphone, hydrocodone, or hydro-morphone. The validity and reliability of sweat patches for drug testing has been judicially confirmed.

While the commercially available dermal patches for drug and alcohol testing claim to be wearable for up to 7 to 10 days, in practice patch adhesion to the skin fails much sooner.

### SUMMARY

This disclosure provides a sweat patch with improved adhesion under common wear conditions, such as profuse sweat or immersion in water, for a period as long as 14 to 21 days or more. The improved wear time is achieved using two adhesives in different regions to secure the sweat patch to the subject. One adhesive secures a central region including the collection pad and an area immediately surrounding the collection pad to the subject's skin, while the second, stronger adhesive secures an outer ring between the central region and the periphery. The second adhesive provides greater adhesion to the skin, thereby providing a longer wear time and greater analyte collection integrity. The strength and durability of the second adhesive also provides one anti-tampering feature. Other anti-tampering features may also be provided.

In a first embodiment, a sweat patch, includes a collection pad and an adhesion pad overlying the collection pad. When applied to skin of a subject, the adhesion pad secures the collection pad in contact with the skin. A first adhesive secures a central region of the adhesion pad surrounding the collection pad to the skin. A second adhesive secures a peripheral region surrounding the central region to the skin. The second adhesive is preferably more durable than the first adhesive. The adhesion pad may comprise a first medical tape within the central region and a second medical tape within the peripheral region and overlapping an outer edge of the first medical tape.

In a second embodiment, a dermal patch for collecting analytes expressed through skin of a subject by perspiration comprises: a collection pad; a first medical tape overlying the collection pad and a first region surrounding the collection pad; and a second medical tape overlapping outer edges of the first medical tape and extending to a periphery of the dermal patch. The second medical tape provides more durable adhesion of the dermal patch to the skin than the first medical tape. For distribution, the dermal patch may further comprise a release liner adjacent to exposed, adhesive-covered surfaces of the first and second medical tapes.

Other technical features may be readily apparent to one skilled in the art from the following figures, descriptions, and claims.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIGS. 1A through 1E depict the structure of a sweat patch with improved adhesion and anti-tampering feature(s) in accordance with one embodiment of the present disclosure;

FIG. 2 depicts the structure of a sweat patch with improved adhesion and anti-tampering feature(s) in accordance with another embodiment of the present disclosure;

FIGS. 3 and 4 depict the structure of a sweat patch with improved adhesion and anti-tampering feature(s) in accordance with various embodiments of the present disclosure; and

FIG. 5 depicts the structure of a sweat patch with improved adhesion and anti-tampering feature(s) in accordance with a further embodiment of the present disclosure.

### DETAILED DESCRIPTION

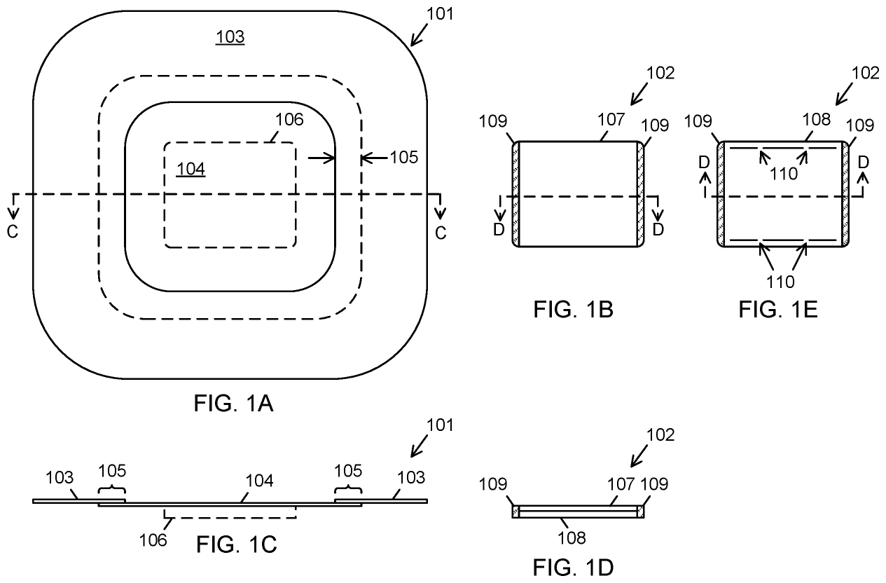

FIGS. 1A through 5, described below, and the various embodiments used to describe the principles of the present disclosure are by way of illustration only and should not be construed in any way to limit the scope of the disclosure. Those skilled in the art will understand that the principles of the present disclosure may be implemented in any type of suitably arranged device or system.

In the present disclosure, improved wear time and anti-tampering is achieved based on the adhesive used to secure the sweat patch to the subject. In one example described in further detail below, two adhesives are used: one adhesive secures regions of the sweat patch immediately surrounding the collection pad to the subject's skin, while a second adhesive secures an outer ring of the sweat patch to the subject's skin. The second adhesive provide greater adhesion to the skin under common conditions, such as profuse sweat by the subject (e.g., due to ambient temperature and/or exercise) or immersion in water while showering/bathing or engaging in watersports. The improved adhesion provides a longer wear time for the sweat patch without edges of the sweat patch coming loose from the subject's skin, thereby also protecting the integrity of the sweat patch with respect to collection of analytes from body fluid rather than from material seeping under the sweat patch edges. The improved adhesion consistently and reliably allows for wear in excess of a week, and as long as 14 to 21 days or more. Two different materials coated with the second adhesive are described herein, with one providing longer wear time than the other—although both improve wear time over commercially available sweat patches. The improved adhesion also provides one anti-tampering feature, since removal of the portion of the sweat patch secured by the second adhesive will inherently deform the sweat patch in one or more visually apparent manners. Other anti-tampering features may also be provided.

FIGS. 1A through 1E depict the structure of a sweat patch with improved adhesion and anti-tampering feature(s) in accordance with one embodiment of the present disclosure. FIG. 1A is a top plan view of an adhesion pad 101 portion of the sweat patch 100. FIG. 1B is a top plan view of a collection pad 102 portion of the sweat patch 100, while FIG. 1E is a bottom plan view. FIG. 1C is a diagrammatic cross-sectional view of FIG. 1A taken at section line C-C, and FIG. 1D is a diagrammatic cross-sectional view of FIGS. 1B and 1E taken at section line D-D. FIGS. 1A through 1E are not drawn to scale, and in particular the relative thicknesses depicted in cross-sectional views are exaggerated for purposes of clarity. The embodiment of the sweat patch 100 illustrated in FIGS. 1A through 1E is for illustration only. FIGS. 1A through 1E do not limit the scope of this disclosure to any particular implementation of a dermal patch.

Sweat patch 100 includes an adhesion pad 101 and a collection pad 102. In the example shown, adhesion pad 101 includes an upper layer 103 and a lower layer 104. As illustrated, upper layer 103 may be a hollow, generally rectangular shape with rounded corners, with overall dimensions of approximately 3.5 inches by 3.75 inches and with the hollow center having dimensions of approximately 1.75 inches by 2.0 inches. Upper layer 103 may comprise a spunlace non-woven tape including a polyester backing with the underside (in the orientation shown in FIG. 1C) coated with a pressure sensitive acrylic adhesive. Upper layer 103 may comprise, for example, 3M Medical Tape 4076 or 3M Medical Tape 4077, both available from 3M Corporation. Lower layer 104 may also be a generally rectangular shape with rounded corners, with dimensions of approximately 2.5 inches by 2.25 inches. The adhesive coating on upper layer 103 comprises the second adhesive with improved adhesion characteristics discussed above. Lower layer 104 may comprise a thin, single sided polyurethane tape that is breathable while providing a liquid barrier, with the underside (in the orientation shown in FIG. 1C) coated with a pressure sensitive acrylic adhesive. Lower layer 104 may comprise, for example, 3M Medical Tape 9834, also available from 3M Corporation. The adhesive coating on lower layer 104 comprises the first adhesive in the region immediately surrounding the collection pad 102 discussed above. Lower layer 104 is preferably somewhat transparent, or at least translucent. As shown in FIGS. 1A and 1, upper layer 103 and lower layer 104 overlap in region 105. The region 106 of the adhesion pad 101 that is occupied by the adjacent collection pad 102 is shown in phantom in FIGS. 1A and 1C.

Collection pad 102 includes a base layer 107 and a collection material layer 108. In the example shown, base layer 107 is generally rectangular shape rounded corners, with dimensions of approximately 1.0 inch by 1.25 inches. Base layer 107 may be, for example, Unitherm 100 polyester paper. Collection material layer 108 in the example shown is generally rectangular with rounded corners, with dimensions of approximately 1.0 inch by 1.25 inches. Collection material layer 108 may be, for example, a cellulose fiber pad such as Cytiva grade CF12 available from the Whatman Lab Filtration Products division of Danaher Corporation. Base layer 107 and collection material layer 108 are secured to each other by weld regions 109 along opposing edges thereof. Collection pad 102 may also include feature(s) such as breaks (“wings”) 110 depicted in FIG. 1E to facilitate separation of collection material layer 108 from base layer 107, after sweat patch 100 is removed from a subject, for testing of analytes within the body fluid collected during wear.

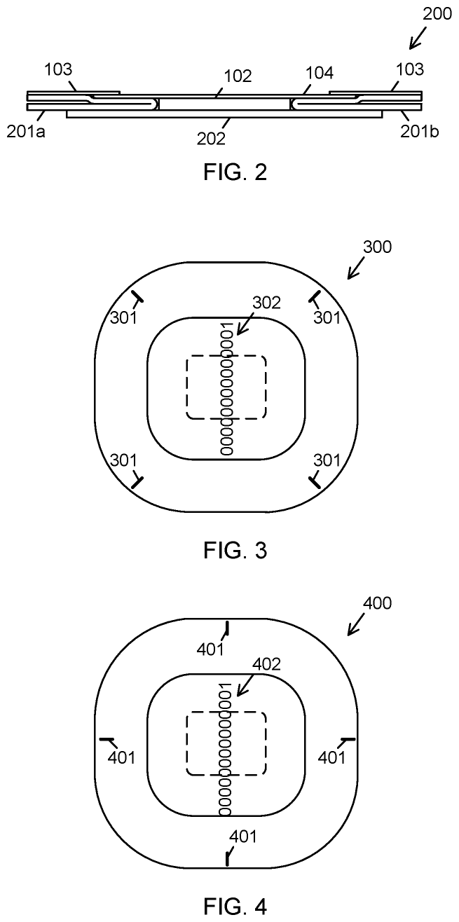

FIG. 2 depicts the structure of a sweat patch with improved adhesion and anti-tampering feature(s) in accordance with another embodiment of the present disclosure. FIG. 2 is a diagrammatic cross-sectional view analogous to FIG. 1C. FIG. 2 is not drawn to scale, and in particular the relative thicknesses depicted are exaggerated for purposes of clarity. The embodiment of the sweat patch 200 illustrated in FIG. 2 is for illustration only. FIG. 2 does not limit the scope of this disclosure to any particular implementation of a dermal patch.

FIGS. 1A through 1E collectively illustrate the structure of sweat patch 100 (by its major components: adhesion pad 101 and collection pad 102) during use—that is, when applied to and while being worn by a subject. FIG. 2, on the other hand, depicts a sweat patch 200 before application—that is, in a form suitable for distribution. Sweat patch 200 includes the same adhesion pad 101 (depicted by upper and lower layers 103, 104) and collection pad 102 structure(s) and material(s) as depicted and described for sweat patch 100. However, sweat patch 200 also includes release liners 201a, 201b and 202. Release liners 201a, 201b and 202 are slick surface paper(s) that may be readily removed from the adhesive(s) coating the surfaces of upper layer 103 and lower layer 104 of adhesion pad 101. Silicone coated paper and other suitable alternatives are known in the art. Release liners 201a, 201b are folded, as shown, and are overlapped by portions of release liner 202.

During application to a subject, release liner 202 is removed from the remainder of the structure illustrated in FIG. 2 and the sweat patch 200 is position over a suitable skin surface, such as on the subject's shoulder or upper arm. The exposed portion of the upper layer 103 and lower layer 104 of adhesion pad 101 are pressed onto the skin surface, holding collection pad 102 between the skin and the adhesion pad 101. Release liners 201a and 201b are then removed from the underlying areas of adhesion pad 101 pressed onto the skin surface.

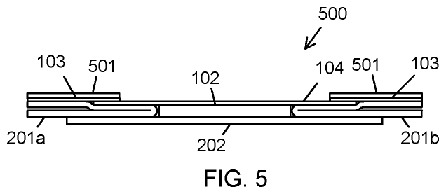

FIGS. 3 and 4 depict the structure of a sweat patch with improved adhesion and anti-tampering feature(s) in accordance with various embodiments of the present disclosure. FIGS. 3 and 4 are top plan views analogous to FIG. 1A. FIGS. 3 and 4 are not drawn to scale. The embodiments of the sweat patch 300, 400 illustrated in FIGS. 3 and 4, respectively, are for illustration only. FIGS. 3 and 4 do not limit the scope of this disclosure to any particular implementation of a dermal patch.

Sweat patches 300, 400 each include the same adhesion pad 101 and collection pad 102 structure(s) and material(s) as depicted and described for sweat patch 100. However, sweat patches 300, 400 include anti-tampering slits 301, 401, respectively, through the upper layer 103 of the adhesion pad 101, proximate to a periphery of the sweat patch. Slits 301, 401 make efforts at removal of the sweat patch even more visually evident, since such efforts will generally result in tearing of the upper layer 103 material. As apparent from FIGS. 3 and 4, the number and location(s) of anti-tampering slits through the upper layer 103 of the adhesion pad may be varied.

A unique patch identifier 302, 402 may be included over the collection pad and portions of the adhesion pad. In the example, shown, the patch identifier 302, 402 is a serial number printed (e.g., by ink jet) on a surface of the sweat patch 300, 400. Alternatively, the patch identifier 302, 402 could include (in lieu of or in addition to the serial number) a bar code, a quick response (QR) code, or a similar graphic code.

FIG. 5 depicts the structure of a sweat patch with improved adhesion and anti-tampering feature(s) in accordance with a further embodiment of the present disclosure. FIG. 5 is a diagrammatic cross-sectional view analogous to FIG. 1C and FIG. 2. FIG. 5 is not drawn to scale, and in particular the relative thicknesses depicted are exaggerated for purposes of clarity. The embodiment of the sweat patch 500 illustrated in FIG. 5 is for illustration only. FIG. 5 does not limit the scope of this disclosure to any particular implementation of a dermal patch.

Like FIG. 2, FIG. 5 depicts the structure of a sweat patch 500 before application, in a form suitable for distribution. Sweat patch 500 includes the same adhesion pad 101 (formed of upper and lower layers 103, 104) and collection pad 102 structure(s) and material(s) as depicted and described for sweat patch(es) 100 and 200. Sweat patch 500 also includes the same release liners 201a, 201b and 202 as the embodiment of FIG. 2. However, sweat patch 500 also includes an additional adhesion layer 501 overlying adhesion layer 101. However, adhesion layer 501 is a single layer of material, and may be either 3M Medical Tape 4076 or 3M Medical Tape 4077. For example, if the upper layer 103 of adhesion pad 101 is 3M Medical Tape 4076, adhesion layer 501 may be 3M Medical Tape 4077. The additional adhesion layer 501 improves the longevity of wear for the sweat patch 500.

It may be advantageous to set forth definitions of certain words and phrases used throughout this patent document. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Although the present disclosure has been described with exemplary embodiments, various changes and modifications may be suggested to one skilled in the art. It is intended that the present disclosure encompass such changes and modifications as fall within the scope of the appended claims. None of the description in this application should be read as implying that any particular element, step, or function is an essential element that must be included in the claims scope. The scope of patented subject matter is defined by the claims.

What is claimed is:

1. A sweat patch, comprising:
a collection pad; and
an adhesion pad overlying the collection pad and, when applied to skin of a subject, securing the collection pad in contact with the skin,
wherein a first adhesive secures a central region of the adhesion pad surrounding the collection pad to the skin, and
wherein a second adhesive secures a peripheral region surrounding the central region to the skin.

2. The sweat patch of claim 1, wherein the second adhesive is more durable than the first adhesive.

3. The sweat patch of claim 1, wherein the first and second adhesives are medical grade adhesives.

4. The sweat patch of claim 1, wherein the adhesion pad comprises a first medical tape within the central region and a second medical tape within the peripheral region and overlapping an outer edge of the first medical tape.

5. The sweat patch of claim 4, further comprising:
a third medical tape over the second medical tape, the third medical tape having a same shape as the second medical tape.

6. The sweat patch of claim 5, wherein the third medical tape is a medical tape different from the first medical tape and the second medical tape.

7. The sweat patch of claim 5, wherein the third medical tape is more durable than the first medical tape.

8. The sweat patch of claim 1, further comprising:
an adhesion layer over the adhesion pad.

9. The sweat patch of claim 8, wherein the adhesion layer is a medical grade adhesive.

10. The sweat patch of claim 8, wherein the adhesion layer has a same shape as the second adhesive.

11. The sweat patch of claim 8, wherein the adhesion layer is a medical tape.

12. A dermal patch for collecting analytes expressed through skin of a subject by perspiration, the dermal patch comprising:
a collection pad;
a first medical tape overlying the collection pad and a first region surrounding the collection pad; and
a second medical tape overlapping outer edges of the first medical tape and extending to a periphery of the dermal patch,
wherein the second medical tape provides more durable adhesion of the dermal patch to the skin than the first medical tape.

13. The dermal patch of claim 12, further comprising:
a release liner adjacent to exposed, adhesive-covered surfaces of the first and second medical tapes.
